SEQUENCE LISTING (18 sequences)

SEQ ID NO: 1 - protein, 72 aa, Artificial Sequence
  The modified Chemokine Peptide, hG31P, synthesized from the laboratory
GSKELRCQCIRTYSKPFHPKFIKELRVIESPPHCANTEIIVKLSDGRELCLDPKENWVQRVVEKFLKRAENS

SEQ ID NO: 2 - protein, 72 aa, Homo sapiens
SAKELRCQCIKTYSKPFHPKFIKELRVIESGPHCANTEIIVKLSDGRELCLDPKENWVQRVVEKFLKRAENS

SEQ ID NO: 3 - protein, 74 aa, Homo sapiens
GASVATELRCQCLQTLQGIHPKNIQSVNVKSPGPHCAQTEVIATLKNGRKACLNPASPIVKKIIEKMLNSDKSN

SEQ ID NO: 4 - protein, 74 aa, Homo sapiens
GAPLATELRCQCLQTLQGIHLKNIQSVKVKSPGPHCAQTEVIATLKNGQKACLNPASPMVKKIIEKMLKNGKSN

SEQ ID NO: 5 - protein, 74 aa, Homo sapiens
GASVVTELRCQCLQTLQGIHLKNIQSVNVRSPGPHCAQTEVIATLKNGKKACLNPASPMVQKIIEKILNKGSTN

SEQ ID NO: 6 - protein, 73 aa, Homo sapiens
AVLRELRCVCLQTTQGVHPKMISNLQVFAIGPQCSKVEVVASLKNGKEICLDPEAPFLKKVIQKILDGGNKEN

SEQ ID NO: 7 - protein, 74 aa, Homo sapiens
SAVLTELRCTCLRVTLRVNPKTIGKLQVFPAGPQCSKVEVVASLKNGKQVCLDPEAPFLKKVIQKILDSGNKKN

SEQ ID NO: 8 - protein, 74 aa, Homo sapiens
SDLYAELRCMCIKTTSGIHPKNIQSLEVIGKGTHCNQVEVIATLKDGRKICLDPDAPRIKKIVQKKLAGDESAD

SEQ ID NO: 9 - protein, 73 aa, Artificial Sequence
  Artificial ELR-CXC chemokine
SAKELRCQACIRTYSKPFHPKFIKELRVIESGPHCANTEIIVKLSDGRELCLDPKENWVQRVVEKFLKRAENS

SEQ ID NO: 10 - protein, 72 aa, Artificial Sequence
  The modified ELR-CXC Chemokine Peptide synthesized from the laboratory
SAKELRCQCIKTYSKPLHPKFIKELRVIPASQFCANTEIIVKLSDGRELCLDPKENWVQRVVEKFLKRAENS

SEQ ID NO: 11 - protein, 71 aa, Artificial Sequence
  The modified ELR-CXC Chemokine Peptide synthesized from the laboratory
SAKELRCCIRTYSKPLHPKFIKELRVIPASQFCANTEIIVKLSDGRELCLDPKENWVQRVVEKFLKAAENS

SEQ ID NO: 12 - protein, 72 aa, Artificial Sequence
  The modified ELR-CXC Chemokine Peptide, IL8-17LIP10, synthesized from the laboratory
GSKELRCQCIRTYSKPLHPKFIKELRVIPASQFCANTEIIVKLSDGRELCLDPKENWVQRVVEKFLKRAENS

SEQ ID NO: 13 - DNA, 21 bp, Artificial Sequence
  The forward primer sequence for Homo spains CXCL8. The nucleic acid primer sequence synthesized from the laboratory.
gagcactccataaggcacaaa

SEQ ID NO: 14 - DNA, 18 bp, Artificial Sequence
  The reverse primer sequence for Homo spains CXCL8. The nucleic acid primer sequence synthesized from the laboratory.
atggttccttccggtggt

SEQ ID NO: 15 - DNA, 20 bp, Artificial Sequence
  The forward primer sequence for Homo spains CXCR1. The nucleic acid primer sequence synthesized from the laboratory.
gaccaacatcgcagacacat

SEQ ID NO: 16 - DNA, 20 bp, Artificial Sequence
  The reverase primer sequence for Homo spains CXCR1. The nucleic acid primer sequence synthesized from the laboratory.
tgcttgtctcgttccacttg

SEQ ID NO: 17 - DNA, 25 bp, Artificial Sequence
  The forward primer sequence for Homo spains CXCR2. The nucleic acid primer sequence synthesized from the laboratory.
ggctaagcaaaatgtgatatgtacc

SEQ ID NO: 18 - DNA, 20 bp, Artificial Sequence
  The forward primer sequence for Homo spains CXCR2. The nucleic acid primer sequence synthesized from the laboratory.
caaggttcgtccgtgttgta

(19) United States
(12) Patent Application Publication
(10) Pub. No.: US 2018/0125938 A1
(43) Pub. Date: May 10, 2018

(54) MODIFIED CHEMOKINE PEPTIDE

(71) Applicant: Rise Technology Co., Ltd., Apia (WS)

(72) Inventors: Jya-Wei CHENG, Tainan (TW); Hsi-Tsu CHENG, Tainan (TW); Hui-Yuan YU, Tainan (TW)

(21) Appl. No.: 15/569,033

(22) PCT Filed: Jun. 3, 2015
(86) PCT No.: PCT/CN2015/080725

§ 371 Date: Oct. 24, 2017

(51) Int. Cl. A61K 38/20; C07K 14/54; A61P 35/00

(52) U.S. Cl. CPC A61K 38/20; A61P 35/00; C07K 14/54

(57) ABSTRACT

The present invention provides a modified chemokine peptide, comprising (a) an “ELR” characteristic sequence which is situated at the N-terminus of the modified chemokine peptide, (b) a “PASQF” characteristic sequence which is neighbored to the upstream of the third cysteine counted from N-terminus of the chemokine peptide, and (c) a modification at the 17^{th} position counted from the N-terminus of the modified chemokine peptide. Additionally, the modified chemokine peptide can be used to treat cancer and inhibit tumor growth.

### CROSS-REFERENCE TO RELATED APPLICATIONS

This is a National Phase application filed under 35 U.S.C. 371 as a national stage of PCT/CN2015/080725, filed Jun. 3, 2015, the content of which is hereby incorporated by reference in its entirety.

### FIELD OF THE INVENTION

The present invention relates to a modified chemokine peptide capable of being a therapeutic antagonist. In particular, the present invention relates to a modified chemokine for treating cancer and inhibiting tumor growth.

### BACKGROUND OF THE INVENTION

Chemokines are a group of inducible, secretory, structurally and related small molecules (approximately 8 to 14 kD). Chemokine is divided intofour subfamilies, such as CXC, CC, CX3C and C. Chemokines are also grouped into two main functional subfamilies: “homeostatic chemokines” and “inflammatory chemokines”.

Chemokine usually has three β-sheets in its structure, and has a α-helix at C terminal and more than 2 conserved cysteines at N-terminus. Chemokines have been categorized into four subfamilies (CXC, CC, CX3C and XC) based on the sequence containing the first two cysteines(Cys) at N-terminus. Among the four types, CXC and CC are the two major subfamilies while XC and CX3C are minor subfamilys. Chemokine biological activities are mediated by chemokine receptors. Chemokine receptors are G protein-coupled seven-transmembrane signaling receptors. The family of chemokine receptors consists of 18 members divided into several classes according to their ligands, chemokines, which are classified based on the spatial arrangement of cysteine residues in their amino terminus: 10 CC chemokine receptors (CCR1-10), 6 CXC chemokine receptors (CXCR1-6), 1 CX3C chemokine receptor (CX3CR1), and 1 C chemokine receptor (XCR1). Further, some chemokine receptors, such as CXCR2, CCR1, CCR2, CCR3 or CCR5, have multiple ligands, while others including CXCR4, CXCR5, CXCR6, CCR8 or CCR9 are specific receptors for one single ligand.

For instance, ELR-CXC chemokine with glutamate (E)-leucine (L)-Arginine (R) characteristic sequence (ELR characteristic sequence) is referred to a protein having the amino acid sequence of ELR-CXC characteristic at N-terminus, and X would be the amino acid having polarity with or without charge. ELR-CXC chemokine always means CXCL1, CXCL2, CXCL3, CXCL5, CXCL6, CXCL7, CXCL8. Their receptors are CXCR1 and CXCR2, and they mainly target to neutrophils. ELR-CXC chemokine can promote the accumulation and activation of neutrophils. Thus, this type of ELR-CXC chemokine plays an important role in the generation of the board-ranged acute and chronic inflammation diseases. These inflammations include psoriasis and rheumatoid arthritis.

Additionally, ELR-CXC chemokine is associated with angiogenesis accompanied upon tumor development, and its inductive mechanism is the activation generated by conjugating this type of chemokine, especially referring to CXCL8, with CXCR1 and CXCR2 on the endothelial cells (ECs). At present, it is proved that many different types of tumors are able to secret ELR-CXC chemokines, and the tumors overexpressing these chemokines would be associated with poor prognosis.

In other word, it is a practicable strategy that the prosurvival, proliferation, metastasis signals following CXCR1 or/and CXCR2 activation could be inhibited via administering antagonist of CXCR1 or CXCR2 competitive to ELR-CXC chemokine. Thus, blockade of the chemokine receptor CXCR1 or/and CXCR2 would be indicative of an increased well treatment. Therefore, it is a very urgent and important issue that how to develop antagonists for these chemokine receptors or/and chemokine-analogous proteins quickly, and reduce the probability of clinical failure for treating various disease (e.g., cancer).

It is therefore attempted by the applicant to deal with the above situation encountered in the prior art.

### SUMMARY OF THE INVENTION

Cancer is the most popular disease cause of death in developed countries. If cancer is diagnosed at an early stage, it is more likely to be treated successfully. Although there has been considerable progress in the diagnosis and treatment of cancer, these drugs are either causing serious side effects or ineffective. Therefore, a novel method or a novel composition for treating cancer or preventing cancer is needed.

In order to solve the above-mentioned problems, according to one embodiment of the present invention, there is provided a modified chemokine for inhibiting tumor growth and treating cancer.

The present invention provides a modified chemokine peptide comprising a peptide sequence. The N-terminus of the peptide sequence comprises two characteristic sequences. One characteristic sequence is a “(a) Glutamate (E)-Leucine (L)-Arginine (R) sequence” which is situated at the N-terminus of the modified chemokine peptide. Another characteristic sequence is a “(b) Proline (P)-Alanine (A)-Serine (S)-Glutamine (Q)-Phenylalanine (F) sequence” which is neighbored to the upstream of the third cysteine (C) counted from N-terminus of a chemokine peptide. The modified chemokine peptide comprise (a) characteristic sequence, (b) characteristic sequence, and a modified position. The above-mentioned modified position is situated at the 17^{th}, 12^{th}, or 13^{th} position counted from the N-terminus of the modified chemokine peptide.

In one embodiment, the phenylalanine (F) residue at position 17^{th} is substituted with leucine (L).

In one embodiment, an un-modified precursor of the modified chemokine peptide is originated from a source chemokine. Only one amino acid residue is located between the first two cysteines at N-terminus of the source chemokine, and the amino acid residue has polarity with or without charge.

In one embodiment, the source chemokine peptide is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8 and the combination thereof.

In one embodiment, the modified chemokine peptide is selected from SEQ ID NO: 10, SEQ ID NO: 11, and SEQ ID NO: 12.

The present invention also provides a pharmaceutical composition, comprising the modified chemokine peptide of the present invention and a pharmaceutically acceptable excipient.

In one embodiment, the modified chemokine peptide is used for treating cancer or inhibiting tumor growth.

The present invention further provides a pharmaceutical composition for treating cancer or inhibiting tumor growth, comprising a therapeutically effective amount of a modified chemokine peptide of the present invention and a pharmaceutically acceptable excipient.

In one embodiment, the cancer comprises prostate cancer, breast cancer, uterine cancer, leukemia, ovarian cancer, endometrial cancer, cervical cancer, colorectal cancer, testicular cancer, lymphoma, rhabdomyosarcoma, neuroblastoma, pancreatic cancer, lung cancer, brain tumor, skin cancer, stomach cancer, oral cancer, liver cancer, laryngeal cancer, gallbladder cancer, thyroid cancer, liver cancer, kidney cancer, or nasopharyngeal carcinoma.

Detailed description of the invention is given in the following embodiments with reference to the accompanying drawings.

### BRIEF DESCRIPTION OF THE DRAWINGS

FIG. 1 illustrates the comparison of sequence alignment to the source chemokine peptide sequences (SEQ ID NO: 1 to 9), i.e. the amino acid sequences of ELR-CXC chemokines with high affinity to CXCR1 or/and CXCR2.

FIG. 2 illustrates the comparison of sequence alignment to the amino acid sequences of the modified chemokine peptides (SEQ ID NO: 10 to SEQ ID NO: 12) in the invention. The SEQ ID NO: 12 also means IL8-17LIP10.

FIG. 3 illustrates that the largest amount of cells was migrated in the present of CXCL8.

FIG. 4 illustrates the number of migrated cells. Black bar represents chemotaxi are triggered by CXCL8. Gray bar represents treatment with ELR-CXC chemokine and CXCL8-IP10 both. White bar represents treating with ELR-CXC chemokine and 17LIP10 both. The SEQ ID NO: 12 also means IL8-17LIP10.

FIG. 5 illustrates the tumor volume of treatment group (IL8-17LIP10; SEQ ID NO: 12) and placebo group (saline). Black bar represents the treatment group. Gray bar represents the placebo group.

FIG. 6 illustrates the tumor weight of treatment group (IL8-17LIP10) and placebo group (saline).

FIG. 7 illustrates the microvessel density of treatment group (IL8-17LIP10) and placebo group (saline).

FIG. 8 illustrates the tumor volume after injection of CXCL8-IP10 or saline. Group A was injected 500 μg/kg of CXCL8-IP10 four times weekly. Group B was injected 500 μg/kg of CXCL8-IP10 twice weekly. Group C was injected 250 μg/kg of CXCL8-IP10 twice weekly. Group D was subcutaneously injected 100 μl of saline every day.

FIG. 9A-9B show the images for external appearance of tumor tissues obtained from mice lung. Mice were sacrificed on day 24 after administration with 500 μg/kg i.p. of CXCL8-IP10 four times weekly (FIG. 9A) or saline (FIG. 9B) to obtain the lung tissue. The white nude indicated by arrow was nidus of the cancer which has spread into lung.

FIG. 10A-10B illustrates the expression level of CXCR1 and CXCL8 in cancer cells.

FIG. 11 illustrates the efficacy of the antagonism of the invention by chemotaxis.

FIG. 12 shows the tumor weights which administrate the modified chemokine peptides of the invention with i.p. on xenograft nude mice model, and comparative to the placebo group.

FIG. 13 illustrates the overall survival of the xenograft nude mice administrated the modified chemokine peptides of the invention compared with the control group.

### DETAILED DESCRIPTION OF THE INVENTION

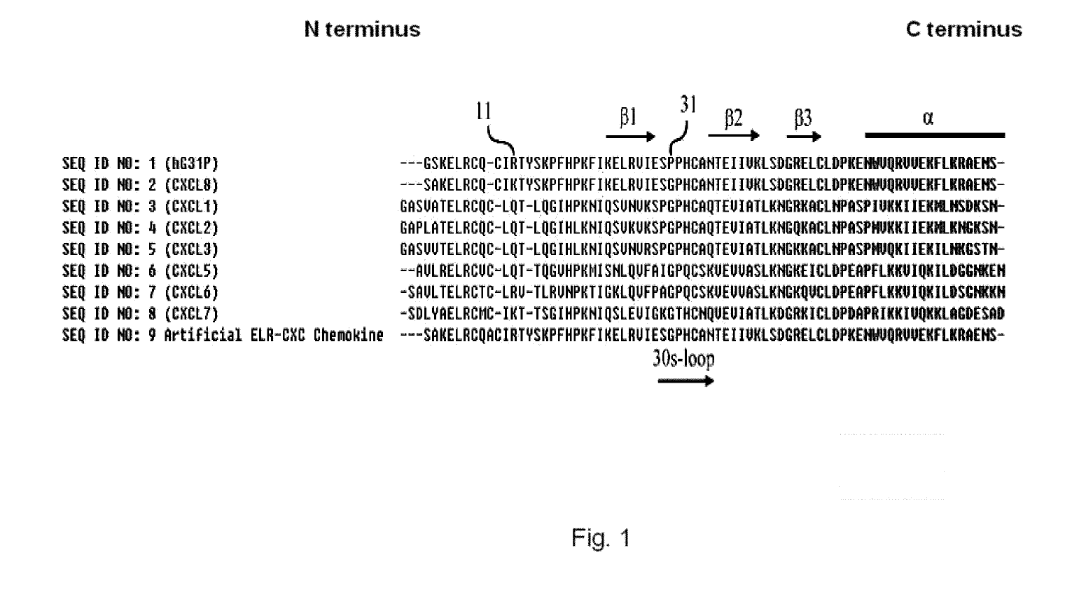

The present invention provides a novel chemokine. The novel chemokine of the present invention is derived from ELR-CXC chemokine or a substance having high binding affinity to CXCR1 or CXCR2, such as CXCL1 (SEQ ID NO: 3), CXCL2 (SEQ ID NO: 4), CXCL3 (SEQ ID NO: 5), CXCL5 (SEQ ID NO: 6), CXCL6 (SEQ ID NO: 7), CXCL7 (SEQ ID NO: 8), CXCL8 (SEQ ID NO: 2), and hG31P (SEQ ID NO: 1). In the preset invention, modification is made in accordance with this type of chemokine in this invention, and it mainly depends on a PASQF characteristic sequence substituted the original 30s-loop region (FIG. 1). This PASQF originally exists in CXCL10 chemokine, which is a non-ELR-CXC chemokine.

In one embodiment, the modified chemokine of the present invention comprises (a) “—N′-Glutamate (E)-Leucine (L)-Arginine (R)” characteristic sequence which is situated at the N-terminus of the modified chemokine peptide, and (b) a “—N′-Proline (P)-Alanine (A)-Serine (S)-Glutamine (Q)-Phenylalanine (F)” characteristic sequence which is neighbored to the upstream of the third cysteine (C) counted from N-terminus of the chemokine peptide. It shall be noted that the modified chemokine of the present invention further comprises a mutation (modification) at the 17^{th} position counted from the N-terminus of the modified chemokine peptide.

In one embodiment, the original amino acid residue at position 17 is phenylalanine (F), and the 17^{th} amino acid residue is substituted to non-phenylalanine (F) amino acid by a single amino acid mutation. In other embodiment, the amino acid residue at position 17 may be substituted to alanine (A), cysteine (C), selenocysteine (U), aspartic acid (D), asparagine (N), glutamic acid (E), glutamine (Q), glycine (G), histidine (H), leucine (L), isoleucine (I), lysine (K), pyrrolysine (O), methionine (M), proline (P), arginine (R), serine (S), threonine (T), valine (V), tryptophan (W), tyrosine (Y), preferably leucine (L). Compared with the source chemokine peptide without any amino acid substitution, the modified chemokines have a higher affinity and could effectively inhibit tumor growth after an amino acid residue substituted at position 17.

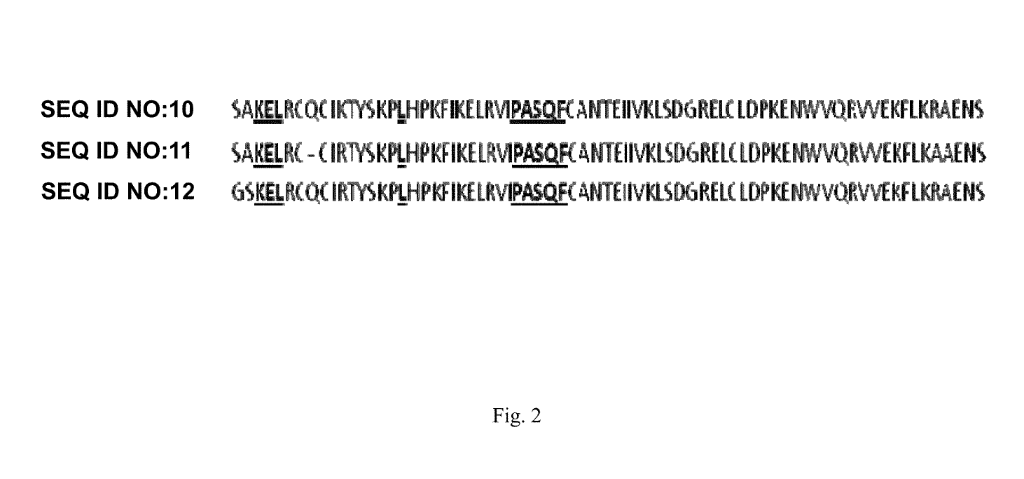

However, the present invention is not limited thereto. According to sequences shown in FIG. 1 and FIG. 2, the modified chemokine of the present invention comprises a peptide sequence. The N-terminus of the peptide sequence comprises a Glutamate (E)-Leucine (L)-Arginine (R) sequence and a Proline (P)-Alanine (A)-Serine (S)-Glutamine (Q)-Phenylalanine (F) sequence.

The Glutamate (E)-Leucine (L)-Arginine (R) sequence is defined as a first characteristic sequence. The first characteristic sequence situated at the N-terminus of the modified chemokine peptide. The modified chemokine peptide has a plurality of modified positions.

The Proline (P)-Alanine (A)-Serine (S)-Glutamine (Q)-Phenylalanine (F) sequence is defined as a second characteristic sequence. The second characteristic sequence is situated between a third Cysteine (Cys³) and an amino acid residue backwards one amino acid position from the third Cysteine. Moreover, in one embodiment, “IP10” also means the second characteristic sequence.

An un-modified precursor of the modified chemokine peptide is originated from a source chemokine peptide. The source chemokine peptide selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8 and the combination thereof.

The first characteristic sequence of the source chemokine peptide from N-terminus is aligned with a 4^{th}, 5^{th}, 6^{th} position of SEQ ID NO: 1 from N-terminus. A first Lysine (K) of the source chemokine peptide from the first ELR sequence toward C-terminus is aligned with a 20^{th} position of SEQ ID NO: 1 from N-terminus. A first modified position is situated at “a first amino acid residue” backwards one amino acid from a first histidine (H) of the source chemokine peptide from N-terminus. The first modified position is aligned with a 17^{1h} position of SEQ ID NO: 1. The 17^{th} amino acid position is phenylalanine (F) from the N-terminus of SEQ ID NO: 1. A second modified position is situated at a first threonine (T) of the source chemokine peptide counted from the first characteristic sequence toward C-terminus. The second modified position is aligned with a 12^{th} position of SEQ ID NO: 1. The 12^{th} amino acid position is Threonine (T) from the N-terminus of SEQ ID NO: 1. A third modified position is situated at “a third amino acid residue” which moves forward one amino acid residue along the source chemokine peptide from the second modified position toward C-terminus. If the third modified position is tyrosine (Y), then the third modified position is aligned with a 13^{th} position counted from the N-terminus of SEQ ID NO: 1.

In one embodiment, the first modified position of the source chemokine peptide could be substituted with leucine (L), valine (V), or isoleucine (I). For example, but not limited, F17L, F17V also mean the first modified position.

In one embodiment, the second modified position of the source chemokine peptide could be substituted with serine (S). For example, but not limited, T12S also means the second modified position.

In one embodiment, the third modified position of the source chemokine peptide could be substituted with leucine (L), phenylalanine (F), Tryptophan (W), or isoleucine (I). For example, but not limited, Y13F, Y13W also mean the third modified position. In other word, if “the third amino acid residue” is Tyrosine (Y), then “the third amino acid residue” could be substituted with phenylalanine (F), or Tryptophan (W). If “the third amino acid residue” is not Tyrosine (Y), then phenylalanine (F), or Tryptophan (W) could be added next from the second modified position toward C-terminus.

Compared with the original chemokine without amino acid substitution, the modified chemokines have a higher affinity and could effectively inhibit tumor growth via an amino acid be substituted.

Optionally, in an exemplary embodiment of the present invention, the modified chemokines of the present invention include, but are not limited to, SEQ ID NO: 10, SEQ ID NO: 11, and/or SEQ ID NO:12(FIG. 2).

Sequences of SEQ ID NO: 10, SEQ ID NO: 11, and SEQ ID NO:12 which can be employed in accordance with the invention are shown hereinbelow:

SEQ. ID NO: 10:
SAKELRCQCIKTYSKPLHPKFIKELRVIPASQFCANTEIIVKLSDGRELC
LDPKENWVQRVVEKFLKRAENS
SEQ. ID NO: 11:
SAKELRCCIRTYSKPLHPKFIKELRVIPASQFCANTEIIVKLSDGRELCL
DPKENWVQRVVEKFLKAAENS
SEQ. ID NO: 12:
GSKELRCQCIRTYSKPLHPKFIKELRVIPASQFCANTEIIVKLSDGRELC
LDPKENWVQRVVEKFLKRAENS

For example, SEQ ID NO: 10 not only includes a N-terminal sequence of ELR-CQC to satisfy the rule of amino acid characteristic sequence of ELR-CXnX, but also has an oligopeptide sequence, Pro-Ala-Ser-Gln-Phe (PASQF). PASQF is a modified sequence substituted for the upstream of the third cysteine counted from N-terminus, and this third cysteine closely neighbors to phenoalanine of PASQF oligopeptide sequence. More importantly, the amino acid residue at 17^{th} position (the first modified position) is leucine, not phenylalanine.

According to sequences shown in FIG. 2, SEQ ID NO: 10, SEQ ID NO: 11, and SEQ ID NO: 12 of the ELR-CXC chemokine analogs have a PASQF modified sequence, which is neighbored to the upstream of the third cysteine (C) counted from N-terminus of a chemokine peptide. Additionally, the amino acid residue at the first modified position of the chemokine in the present invention is substituted by leucine.

For example, SEQ ID NO: 12 represents IL8-17LIP10. “17L” of IL8-17LIP10 means the amino acid residue at the first modified position (17^{th} amino acid position) of the chemokine substituted by leucine. “IP10” of IL8-17LIP10 means the second characteristic sequence (PASQF).

The modified chemokine of the present invention, an analogs and fragments thereof can treat diseases associated with angiogenesis. The angiogenesis associated diseases include, but are not limited to, inflammatory disorders, chronic articular rheumatism and psoriasis, disorders associated with inappropriate or inopportune invasion of vessels such as diabetic retinopathy, neovascular glaucoma, restenosis, and cell proliferation disorder/disease, such as neoplasm and cancer associated disorders (e.g., prostate cancer, breast cancer, uterine cancer, leukemia, ovarian cancer, endometrial cancer, cervical cancer, colorectal cancer, testicular cancer, lymphoma, rhabdomyosarcoma, neuroblastoma, pancreatic cancer, lung cancer, brain tumor, skin cancer, stomach cancer, oral cancer, liver cancer, laryngeal cancer, gallbladder cancer, thyroid cancer, liver cancer, kidney cancer, or nasopharyngeal carcinoma).

The modified chemokine of the present invention can be administered orally, buccally, parenterally, by inhalation spray, rectally, intradermally, transdermally, or topically in dosage unit formulations containing conventional nontoxic pharmaceutically acceptable carriers, adjuvants, and vehicles as desired.

The modified chemokine of the present invention can be administered in a single dose, in multiple doses throughout a 24-hour period, or by continuous infusion. When administered by continuous infusion, the compounds can be supplied by methods well known in the art, such as, but not limited to, intravenous gravity drip, intravenous infusion pump, implantable infusion pump, or any topical routes. Length of treatment will vary depending on many factors, for example, the duration and severity of the angiogenesis condition. Treatment of the subject with the modified chemokine of the present invention alone or in combination with other agents may last until the angiogenesis disappears, or treatment will continue for the life of the subject.

In another embodiment, the present invention provides a pharmaceutical composition for treating cancer and inhibiting tumor. The pharmaceutical composition comprises an effective amount of a modified chemokine of the present invention or analogs thereof and a pharmaceutically acceptable carrier. The pharmaceutically acceptable carrier could include solvent, dispersants, coating, antibacterial/antifungal agents, isotonicity agents, controlled release agents and/or analogues thereof.

Additional specific embodiments of the present invention include, but are not limited to the following:

### Example 1

Cell Chemotaxis (the Inhibitory Effect of Cell Migration Administrated Modified Chemokine Peptides)

Boyden chamber assay was used to evaluate the migration of LMVECs. LMVECs were cultured on HuMedia-EB2 with 2% FCS for 8 hours. Cells were seeded on a polycarbonate membrane (Sigma-Aldrich) coated with 10 μg/ml of fibronectin by a density of 1.2×10⁵ cell/cm². 10 ng/ml of CXCL8, CXCL6, CXCL1, CXCL5, and CXCL8-IP10 or IL8-F17LIP10 was added to the bottom of Boyden chamber, respectively. LMVECs were cultured on Boyden chamber at 37° C. for 4 hours, and then fixed and stain with Diff-Quick (Harleco). The number of migrated cells was counted by HPFs (X200).

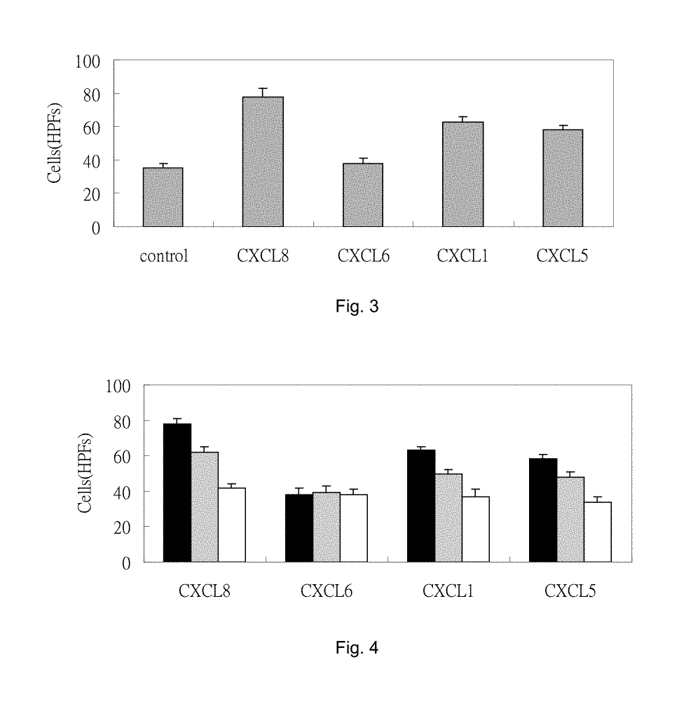

The result represent that a large amount of cell was migrated when present of chemokine CXCL8 (FIG. 3).

In FIG. 4, black bar represents chemokine induction, gray bar represents treat with chemokine and CXCL8-IP10 together, and white bar represents treat with chemokine and IL8-17LIP10 (SEQ ID NO: 12) simultaneously. According to FIG. 4, CXCL8-IP10 and IL8-17LIP10 both had the effect to inhibit the cell migration, and the IL8-17LIP10 has a better inhibiting effect on cell migration than CXCL8-IP10.

### Example 2

The Anti-Tumor Effect in BALB/c Nude Male Mice Bearing Xenograft Tumor Via Administrated Modified Chemokine Peptides

BALB/c Nude male mice (Bltw:NU-Foxn1^{nu}, 4-6 weeks-old) were obtained and maintained in a laminar airflow cabinet under the specific pathogen free conditions. The animals freely accessed to tap water and standard pellet food, and their health was monitored daily. For the nude mouse xenograft assay, the monolayer-cultured GFP-positive PC3 cells (PC-3-GFP) were harvested and inoculated subcutaneously into the right flank of three nude mice with 5×10⁶ cell per mouse. After 2 to 4 weeks, tumor was harvested for implantation. At the end of the experiments, the tumor xenografts from these three mice were reset, sliced (1 mm³ sections), and then implanted to prostate tissues of the recipient nude mice under local anesthesia and sterile surgical conditions. A total of 24 animals received implants. Five days later (day 0), the animals were classified into two groups (12 animals/group) for subcutaneous injection of 100 μl of normal saline (control group) or the sequence of the present invention (SEQ ID NO: 12 (IL8-17LIP10), 0.5 mg/kg) (experimental group) for 24 days. GFP fluorescence images of the growing tumors were captured on day 12, 18, and 24 using a digital camera under the optical configuration of a dissection microscope with 515 nm emission filter. The tumor volumes were calculated using a formula: Volume=(length×width)/2. On day 24, all of mice were sacrificed and GFP fluorescence images of the tumors were captured. Vascular microvessel density was calculated using the formula: Density=microvessel length/tumor area. Tumor samples from each mouse were fixed in 4% paraformaldehyde and embedded in paraffin using standard procedures for subsequent immunohistochemical analyses.

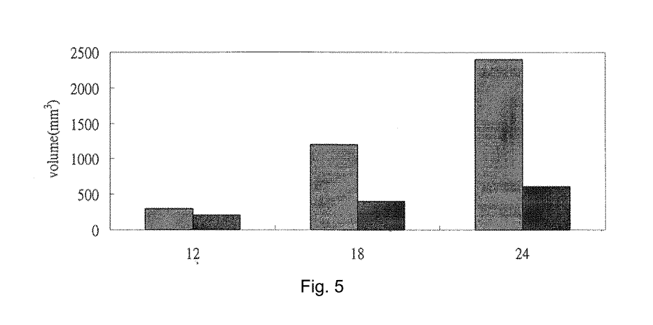

The result shows that the tumor volume of treatment (experimental) group and placebo (control) group on day 12, 18, and 24 (FIG. 5). According to FIG. 5, the sequence (IL8-17LIP10; SEQ ID NO: 12) of the present invention effectively inhibits tumor growth. The tumor volume of treatment group was reduced by more than 5 times compared with placebo group. This apparent inhibition of tumor volume for treatment group compared with placebo group was sustained and became more pronounced over time.

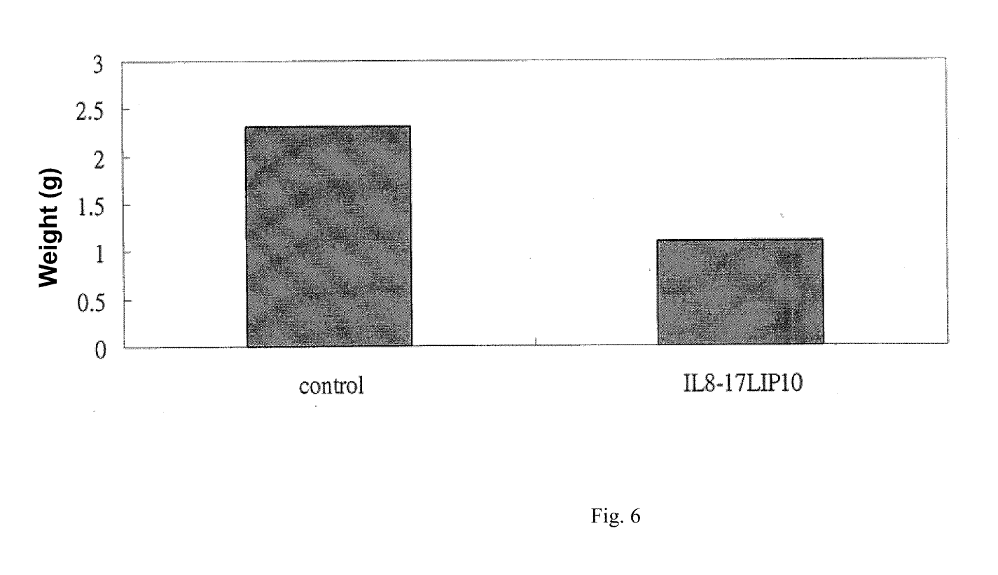

The result shows that the tumor weight of treatment (experimental) group and placebo (control) group on day 24 (FIG. 6). According to FIG. 6, the sequence (IL8-17LIP10; SEQ ID NO: 12) of the present invention effectively inhibits tumor growth, and the tumor weight was decreased by more than 2 times.

### Example 3

Immunohistochemical Analysis for Xenograft Tumor Tissue

Paraffin-embedded prostate cancer xenograft sections were dewaxed and rehydrated into PBS. In detail, the sections were rinsed three times with PBS and heat-treated for 15 min in 10 mM sodium citrate (pH 6.0). The endogenous peroxidase activities were blocked by treatment with 3% hydrogen peroxide for 10 min. The sections were rinsed three times with PBS again, incubated with a protein-blocking solution (5% normal horse serum in PBS, pH 7.5) for 15 min at room temperature, washed with three times with PBS, and then incubated with a mouse monoclonal anti-VEGF antibody (1:50), a rabbit polyclonal anti-NF-κB antibody, or a goat polyclonal anti-CD31 antibody (1:50) for 20 hours at 4° C. After reaction, the sections were washed three times with PBS and incubated with the appropriate dilution of the secondary antibody for 40 min at 37° C. After washing three times with PBS, the sections were incubated with biotinylated goat anti-mouse or anti-rabbit anti-goat poly-immunoglobulin for 30 min in the dark. For color development, the sections were washed with PBS three times, incubated in diaminobenzidine solution for 10 min, and then counterstained with hematoxylin for 1 min (Kollmar et al, 2007). The negative control sections were incubated with PBS instead of the primary antibody. The intensity of the stained sections were measured after converting photographed sections into gray scale (scale 0-255) and represented by integrated optical density (IOD), which was calculated using Image-Pro 6.0 Microsoft. For each group, five mouse tumor xenografts were analyzed. Five photographed sections per tumor, which were chosen randomly in different sections, were used to average gray values of every tumor xenograft (Csillik et al., 2005). Immunohistochemical analyses of CD31 were used to determine microvessel density, and the measurement of microvessel density represents the mean percentage of microvessel area, which was calculated using Image-Pro 6.0 Microsoft.

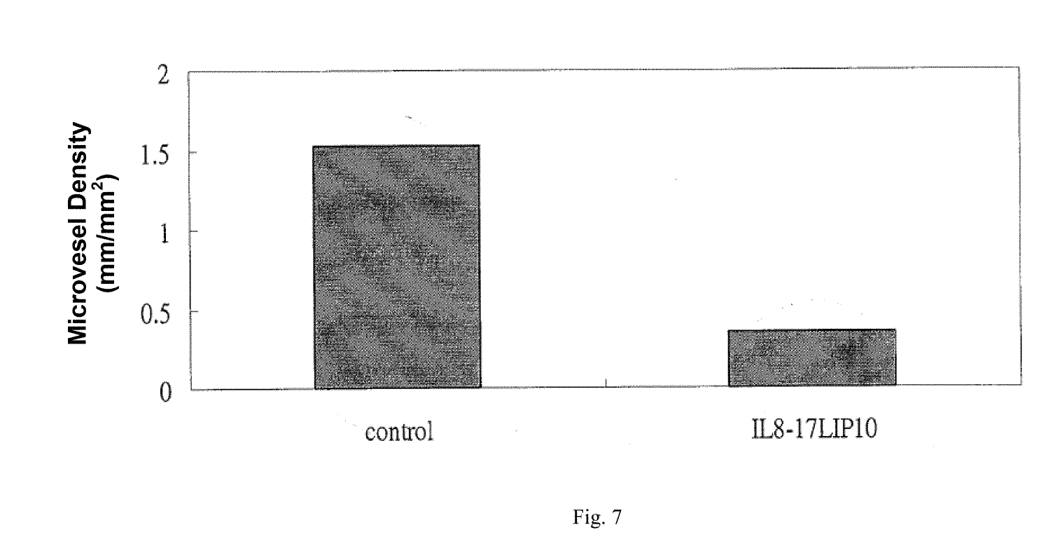

The result shows that the microvessel density of placebo (control) group and treatment (experimental) group (FIG. 7). According to FIG. 7, the sequence (IL8-17LIP10) of the present invention effectively inhibits angiogenesis on prostate cancer xenograft nude mice.

### Example 4

The Anti-Tumor Effect in C57BL/6 Nudemice Bearing Xenografttumor Via Administrated Modified Chemokine Peptides

C57BL/6 nude mice were maintained in a laminar airflow cabinet under specific pathogen. The animals freely accessed to tap water and standard pellet food, and their health was monitored daily. 5×10⁶ cells of LLW2 cells (Lewis lung carcinoma) were injected to right quadrant of three mice with 5×10⁶ cells per mouse. After 2 to 4 weeks, tumor was harvested for implantation. 24 mice were implanted in this example. After 5 days of implantation, 24 mice were classified into four groups (six mice per group). Group A: four times weekly administration of 500 μg/kg of CXCL8-IP10. Group B: twice weekly administration of 500 μg/kg of CXCL8-IP10. Group C: twice weekly administration of 250 μg/kg of CXCL8-IP10. Group D: 100 μl of saline was subcutaneously administrated every day. The tumor volumes were calculated using a formula: Volume=(length×width)/2. On day 24, all of mice were sacrificed and GFP fluorescence images of the tumors were captured. Vascular microvessel density was calculated using the formula: Density=microvessel length/tumor area. Tumor samples from each mouse were fixed in 4% paraformaldehyde and embedded in paraffin using standard procedures for subsequent immunohistochemical analyses.

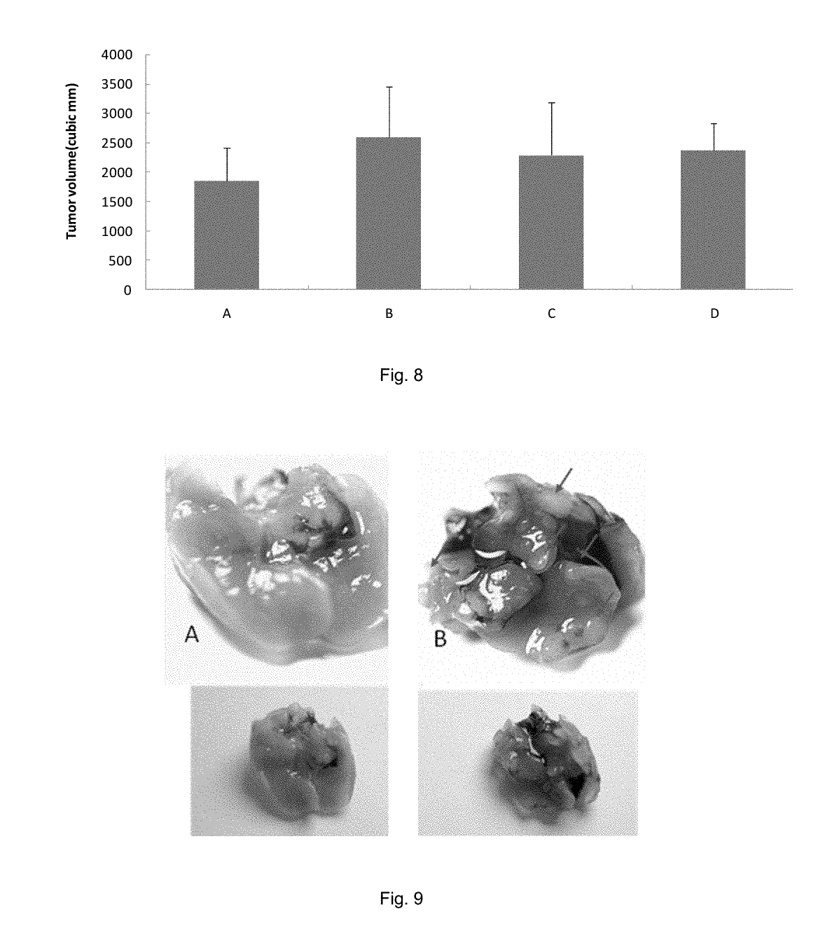

According to FIG. 8, Group A shows the best improvement in tumor growth inhibition. On day 24, all of mice were sacrificed to calculate the tumor volume. Compared to Group D, the tumor volume, which were treated with CXCL8-IP10 (500 ug/kg) for four times per week, was reduced by 30% comparative to Group A. The results indicate that CXCL8-IP10 had significant inhibition of tumor growth.

According to FIG. 9, tumor metastasis was significantly suppressed by CXCL8-IP10. Mice were sacrificed on day 24 to obtain the lung after administration with 500 μg/kg of CXCL8-IP10 four times weekly (FIG. 9A) or saline (FIG. 9B). The white part indicated by arrow was nidus of the cancer which has spread into lung. After administration of CXCL8-IP10, no metastatic nidus occurred in mice lung.

### Example 5

Neutrophil Chemotaxis Assay

The neutrophil chemotaxis was evaluated by an improved Boyden chamber microchemotaxis assay. Leucocytes were collected from peripheral blood by a general concentration gradient. Neutrophils were obtained from the bottom of low concentration gradient area, and erythrocyte contamination was removed by hypotonic lysis. The purified neutrophils (5×10⁶/ml) were suspended in HBSS solution (400 mg/L KCl, 60 mg/L KH₂PO₄, 8000 mg/L, NaCl, 350 mg/L NaHCO₃, 90 mg/LNaH₂PO₄.7H₂O, and 1000 mg/L glucose), and then cultured in Calcein AM (Invitrogen, Stockholm, Sweden) medium at 37° C. for 30 minutes. Chemokine (e.g., 20 ng/ml of CXCL8) was placed on the lower chamber alone or in combination with other antagonists (e.g., IL8-IP10F17L), and the purified neutrophils were placed on the upper chamber. An 5 μm pore-size polycarbonate filter was used to separate the upper and lower chamber. After the cells were cultured in a 5% CO₂ humidified atmosphere at 37° C. for the 30 minutes, the unmigrated cells and filter were removed and the migrated cells were lysed and analyzed using VICTOR3 (Perkin-Elmer, UK, excitation: 485 nm; emission: 530 nm). A percentage of the cell migration was indicated by chemotaxis index (CI) value.CI=(intensity antagonist-intensity HBSS)/(intensity CXCL8−intensity HBSS)×100%, wherein “intensity antagonist” is the cell migration caused by antagonists, “intensity CXCL8” is the cell migration caused by CXCL8, and “intensity HBSS” is the cell migration caused by gravity.

### Example 6

Expression Level of CXCR1/2 and CXCL8 Genes in Tumor Cells

The RNA of cancer cell lines (Table 1) was extracted by TRIzol reagent (Invitrogen, America), respectively using the standard procedures, and the RNA was quantified by NanoDrop®ND-1000 spectrophotometer. A cDNA reverse-transcription kit (Prime Script™ RT reagent kit, Takara, Japan) was used to carry out the reverse transcription of the samples's RNA, and the samples were placed on ice before gene expression analysis. GAPDH was internal control group. RT-PCR reaction was carried out by SYBR (Premix Ex Taq™, Takara, Japan), wherein the primers of CXCL included: 5′-gagcactccataaggcacaaa-3′ (forward) and 5′-atggttccttccggtggt-3′ (reverse) for CXCL8; 5′-gaccaacatcgcagacacat-3′ (forward) and 5′-tgcttgtctcgttccacttg-3′ (reverse) for CXCR1; 5′-ggctaagcaaaatgtgatatgtacc-3′ (forward) and 5′-caaggttcgtccgtgttgta-3′ (reverse) for CXCR2.

The gene expression was calculated by the following formula: gene expression=2^{-ΔΔCt}.

TABLE 1
Tumor cell lines

| | |
|---|---|
| HCC827 | lung adenocarcinoma |
| HCC827GR | HCC827 gefitinib-resistant |
| H1975 | lung adenocarcinoma |
| H2170 | lung squamous cell carcinoma |
| H157 | oral squamous cell carcinoma |
| CT26 | colon carcinoma cell |
| CL1-0 | lung adenocarcinoma |
| CL1-5 | lung adenocarcinoma |
| PC9 | lung adenocarcinoma |
| H3255 | Non small cell lung carcinoma |
| A549 | lung adenocarcinoma |
| H520 | lung squamous cell carcinoma |
| H460 | Non small cell lung carcinoma |

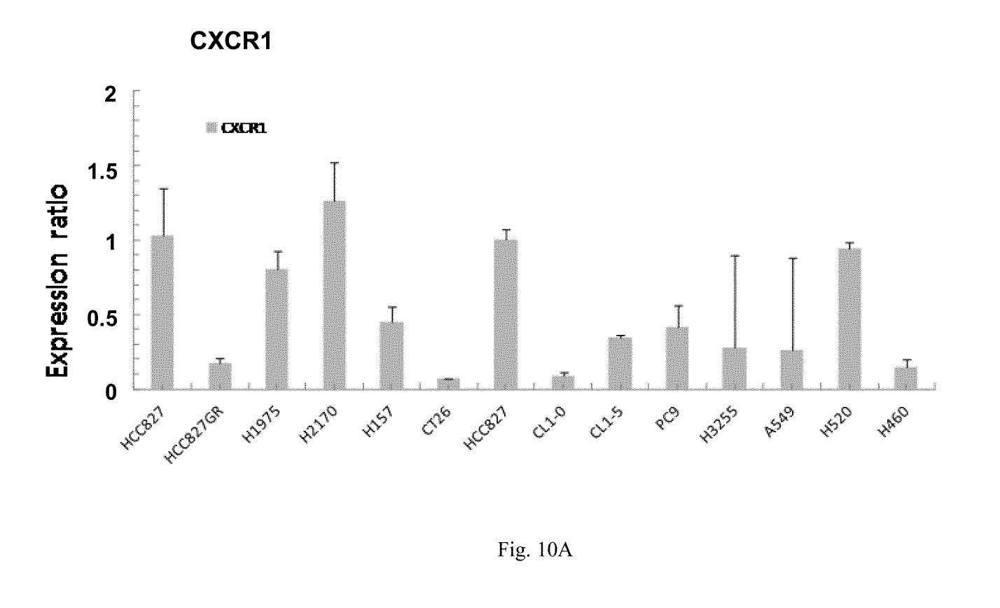

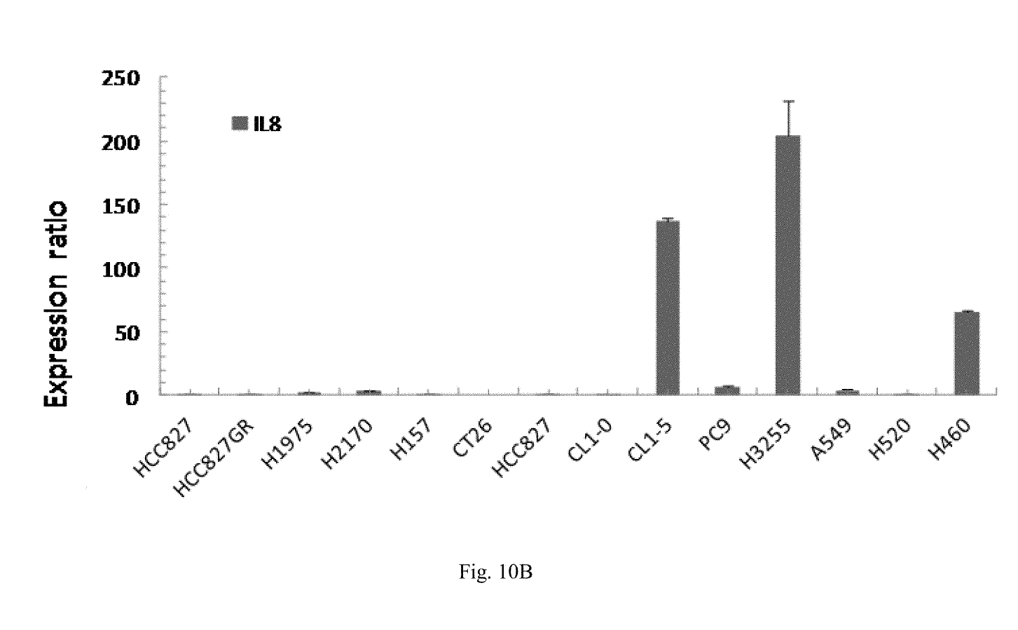

The FIG. 10A-10B illustrates high CXCR1 expression in tumor cells partially by RT-PCR. Many tumor cells highly expressed CXCL8, which is a substance of CXCR1 and CXCR2 receptors. Thus, CXCR1/2 antagonist could be used to inhibit tumor growth and metastasis.

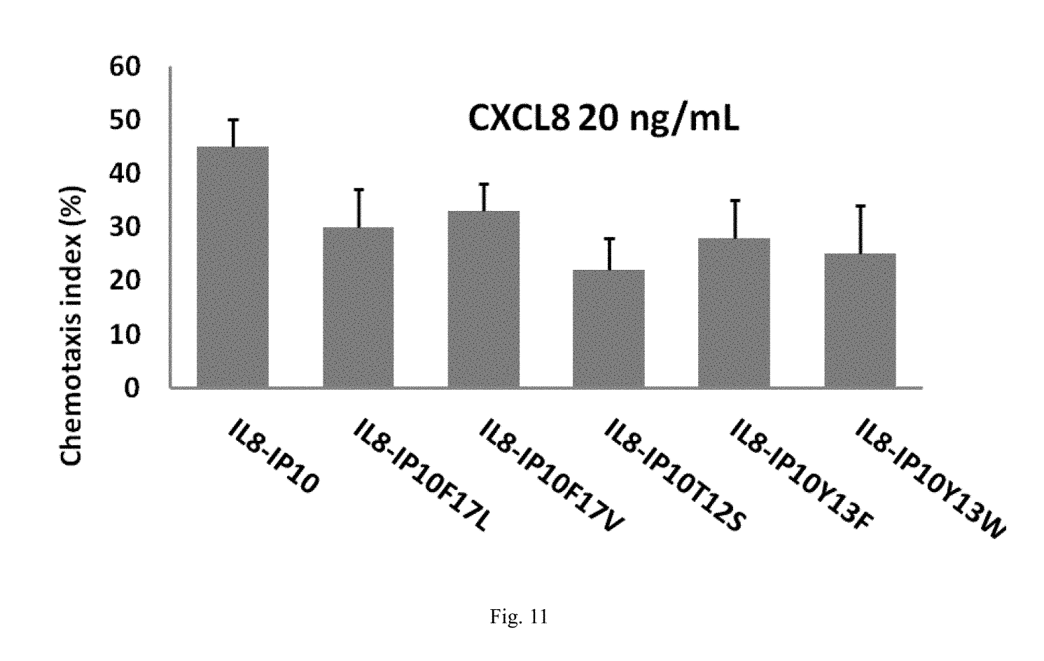

The FIG. 11 illustrates more design of antagonist. The amino acid residue(s) at the first modified position, and/or the second modified position, and/or the third modified position of CXCL8-IP10 peptide was changed to improve the antagonist properties induced by CXCR1/2. These antagonists effectively suppressed the expression of CXCR1/2 receptors, or inhibited tumor growth, drug resistance, metastasis, and angiogenesis associated with high cytokines expression (CXCL1, 2, 3, 5, 6, 7, and 8, etc.) in tumor cells.

### Example 7

The Anti-Tumor Effect and Survival Rate in Nude Mice Bearing Xenografttumor Via Administrated Modified Chemokine Peptides

Nude mice (4- to 6-weeks old) were purchased from National Laboratory Animal Center (Taiwan). PC9 cells were harvested and resuspended at a density of 1×10⁷ cells/ml in sterile phosphate-buffered saline. In the experimental group, PC9 cells were pre-treated with 200 μg/ml modified chemokine peptides which including IL8T12SIP10, CXCL5T12SIP10, IL8T12S, and CXCL5T12S. 1×10⁶ PC9 cells (mixed equal volume matrigel) were injected subcutaneously into the back of the mice (10 mice for control group, IL8T12SIP10 group, and CXCL5T12SIP10 group; otherwise 3 mice for IL8T12S group and CXCL5T12S group). Then, modified chemokine peptides (500 μg/kg) or PBS (control group) were administrated by intraperitoneal injection every 3 days a week. All mice were sacrificed at day 21.

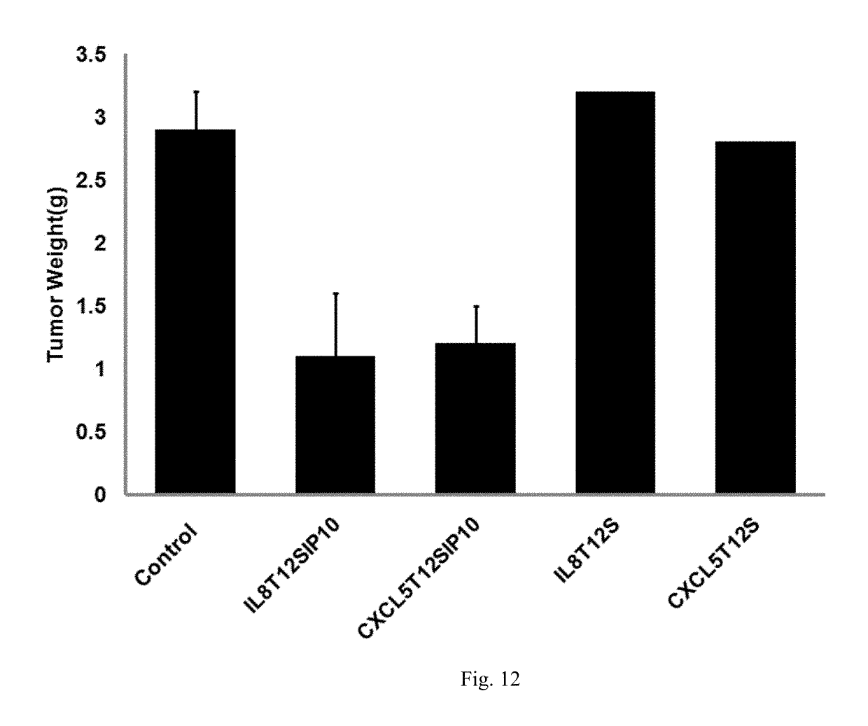

The result shows that the tumor weight of experimental and control groups on day 21 (FIG. 12). According to FIG. 12, the sequences (“IL8T12SIP10” and “CXCL5T12SIP10”) of the present invention can effectively inhibit tumor growth, and the tumor weight was reduced by more than 2 times.

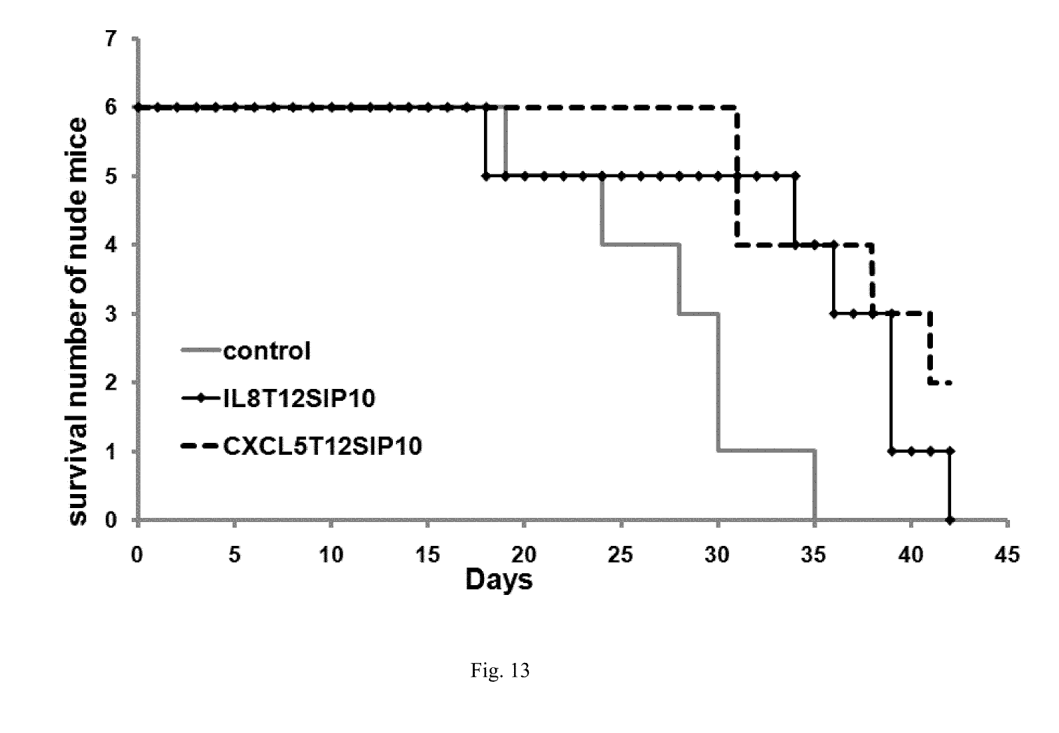

In the meantime, the survival days of nude mice bearing xenograft tumor via administrated modified chemokine peptides, for example, but not limited, “IL8T12SIP10” and “CXCL5T12SIP10”, were obviously longer than the control group (FIG. 13).

As mentioned above, the modified chemokine of the present invention can effectively inhibit tumor growth and angiogenesis and treat cancer.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes, and substitutions are intended in the foregoing disclosures. It will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

1. A modified chemokine peptide comprising a peptide sequence, wherein the N-terminus of the peptide sequence comprises:
(a) a “Glutamate (E)-Leucine (L)-Arginine (R)” sequence is defined as an A characteristic sequence, wherein the A characteristic sequence situated at the N-terminus of the modified chemokine peptide;
(b) a “Proline (P)-Alanine (A)-Serine (S)-Glutamine (Q)-Phenylalanine (F)-Cys” sequence is defined as a B characteristic sequence, wherein the B characteristic sequence is neighbored to the upstream of the third cysteine (C) counted from N-terminus of a chemokine peptide, and
wherein the modified chemokine peptide is consisting of the A characteristic sequence, the B characteristic sequence, and a modified position, wherein the modified position is situated at the 17^{th}, 12^{th}, or 13^{th} position counted from the N-terminus of the modified chemokine peptide.

2. The modified chemokine peptide according to claim 1, wherein the phenylalanine (F) at the 17^{th} position from the N-terminus of the modified chemokine peptide is substituted with leucine (L), valine (V), or isoleucine (I).

3. The modified chemokine peptide according to claim 1, wherein the threonine (T) at the 12^{th} position from the N-terminus of the modified chemokine peptide is substituted with serine (S).

4. The modified chemokine peptide according to claim 1, wherein the tyrosine (Y) at the 13^{th} position from the N-terminus of the modified chemokine peptide is substituted with leucine (L), phenylalanine (F), Tryptophan (W), or isoleucine (I).

5. The modified chemokine peptide according to claim 1, wherein an un-modified precursor of the modified chemokine peptide is originated from a source chemokine peptide, wherein the source chemokine peptide has zero to two one amino acid residue(s) situated between first cysteine and second cysteine from N-terminus, and the amino acid residue(s) has polarity with or without charge when the number of the amino acid residue is one to two.

6. The modified chemokine peptide according to claim 5, wherein the source chemokine peptide is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8 and the combination thereof.

7. The modified chemokine peptide according to claim 1, wherein the modified chemokine peptide is selected from SEQ ID NO: 10, SEQ ID NO: 11, and SEQ ID NO: 12.

8. A pharmaceutical composition comprising a modified chemokine peptide of claim 1 and a pharmaceutically acceptable excipient.

9. The pharmaceutical composition of claim 8, wherein the modified chemokine peptide is used to treat cancer or inhibit tumor growth.

10. A pharmaceutical composition for treating a cancer and inhibiting tumor growth comprising a therapeutically effective amount of a modified chemokine peptide of claim 1 and a pharmaceutically acceptable excipient.

11. The pharmaceutical composition of claim 10, wherein the cancer comprises prostate cancer, breast cancer, uterine cancer, leukemia, ovarian cancer, endometrial cancer, cervical cancer, colorectal cancer, testicular cancer, lymphoma, rhabdomyosarcoma, neuroblastoma, pancreatic cancer, lung cancer, brain tumor, skin cancer, stomach cancer, oral cancer, liver cancer, laryngeal cancer, gallbladder cancer, thyroid cancer, liver cancer, kidney cancer, or nasopharyngeal carcinoma.

12. The pharmaceutical composition of claim 11, wherein the cancer is characterized by cells with a CXCR1/2 expression or a high amount of chemokines consisting of CXCL8, CXCL1, CXCL2, CXCL3, CXCL5, CXCL6 and CXCL7, wherein the cancer is characterized by cancer cells expressed with chemokine receptors.

13. A modified chemokine peptide comprising a peptide sequence, wherein the N-terminus of the peptide sequence comprises:
a Glutamate (E)-Leucine (L)-Arginine (R) sequence is defined as a first characteristic sequence, wherein the first characteristic sequence situated at the N-terminus of the modified chemokine peptide, wherein the modified chemokine peptide has a plurality of modified positions;
a Proline (P)-Alanine (A)-Serine (S)-Glutamine (Q)-Phenylalanine (F) sequence is defined as a second characteristic sequence, wherein the second characteristic sequence is situated between a third Cysteine (Cys³) and an amino acid residue backwards one amino acid position from the third Cysteine,
wherein an un-modified precursor of the modified chemokine peptide is originated from a source chemokine peptide, wherein the source chemokine peptide selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8 and the combination thereof; and
wherein the first characteristic sequence of the source chemokine peptide from N-terminus is aligned with a 4^{th}, 5^{th}, 6^{th} position of SEQ ID NO: 1 from N-terminus, wherein a first Lysine (K) of the source chemokine peptide from the first ELR sequence toward C-terminus is aligned with a 20^{th} position of SEQ ID NO: 1 from N-terminus, wherein a first modified position is situated at a first amino acid residue backwards one amino acid from a first histidine (H) of the source chemokine peptide from N-terminus, wherein the first modified position is aligned with a 17^{th} position of SEQ ID NO: 1, wherein a second modified position is situated at a first threonine of the source chemokine peptide counted from the first characteristic sequence toward C-terminus, wherein the second modified position is aligned with a 12^{th} position of SEQ ID NO: 1, wherein a third modified position is situated at a third amino acid residue forwards one amino acid residue from the second modified position toward C-terminus.

14. The modified chemokine peptide according to claim 13, wherein the first modified position is substituted with leucine (L), valine (V), or isoleucine (I).

15. The modified chemokine peptide according to claim 13, wherein the second modified position is substituted with serine (S).

16. The modified chemokinepeptide according to claim 13, wherein the third modified position is substituted with leucine (L), phenylalanine (F), Tryptophan (W), or isoleucine (I).

17. The modified chemokine peptide according to claim 13, wherein the sequence between first cysteine and second cysteine at N-terminus of the source chemokine peptide has one amino acid residue, and the one amino acid residue has polarity with or without charge.

18. The modified chemokine peptide according to claim 13, wherein the modified chemokine peptide is selected from SEQ ID NO: 10, SEQ ID NO: 11, and SEQ ID NO: 12.

19. A method for treating cancer in a subject, wherein the method comprising administering to said subject having a cancer an effective amount of a modified chemokine peptide of claim 13 and a pharmaceutically acceptable excipient.
